(19) United States
(12) Patent Application Publication
(10) Pub. No.: US 2023/0112979 A1
(43) Pub. Date: Apr. 13, 2023

(54) INFUSION PUMP WITH ALARM MANAGER

(71) Applicant: ICU Medical, Inc., San Clemente, CA (US)

(72) Inventor: Ben Xavier, San Diego, CA (US)

(21) Appl. No.: 17/907,749

(22) PCT Filed: Apr. 6, 2021
(86) PCT No.: PCT/US2021/026057

§ 371 Date: Sep. 29, 2022

Related U.S. Application Data
(60) Provisional application No. 63/006,285, filed on Apr. 7, 2020.

(51) Int. Cl. G08B 29/18; A61M 5/168

(52) U.S. Cl. CPC G08B 29/185; A61M 5/16831; A61M 2205/18; A61M 2205/583; A61M 2205/581; A61M 2205/52; A61M 2205/3561

(57) ABSTRACT

An infusion pump is configured to execute one or more alarm actions according to an alarm protocol. An alarm manager of the infusion pump is configured to receive alarm configuration comprising one or more alarm protocols, each protocol comprising one or more alarm parameters. The alarm manager is also configured to determine the presence of an alarm condition and perform an action associated with the one or more alarm parameters in response to determining the presence of the error condition.

### TECHNICAL FIELD

This disclosure relates to the field of infusion pumps, and particularly to infusion pumps configured to receive and process alarm configuration data to manage alarm local and remote alarm activation to reduce patient stressors and clinician alarm fatigue.

### BACKGROUND

Infusion pumps for infusing one or more fluids into a medical patient are commonplace in modern healthcare environments. Infusion pumps, like many other medical devices, can activate visual and audible indicators as an alarm when the infusion pump detects an error condition. However, in the clinical environment, alarm fatigue or alarm obscurity can result when medical devices alarm too frequently. In addition, audible alarms may cause stress and discomfort to the patient. In many cases, depending upon the type of therapy being delivered (including the type of drug being delivered by an infusion pump) it may not be necessary to activate a particular alarm when an error condition is detected. Therefore, an infusion pump configured with an alarm manager can advantageously control alarm occurrences, which can reduce alarm fatigue and obscurity, and lead to less patient disturbances and stressors when receiving therapy.

### SUMMARY

Various techniques for providing alarm management with an infusion pump are described herein. Although many of the examples are described in the context of a networked hospital environment, the techniques described herein can be applied to any networked or non-networked environment, including but not limited to networks that provide communication between one or more medical devices and a local or remotely located server. Communication may be provided over the hospital network, the Internet, or a combination of both. The infusion pumps described herein sometimes may be other medical devices (instead of or including an infusion pump), or non-medical devices, or any combination thereof. In various embodiments, an infusion pump with alarm management is configured to receive alarm configuration settings, such as from a user or a database, either locally (e.g., at the pump) or remotely (e.g., over a computing network). In some embodiments, the alarm configuration settings are received from or as part of a drug library. The infusion pump is also configured to determine one or more alarm protocols using the alarm configuration settings and optionally also from therapy information of the drug library. The alarm protocol may define one or more actions, alarm attributes, and periods associated with the alarm protocol. Different actions may be taken during each period. The alarm manager is configured to determine whether to activate a visual and/or audible alarm, and whether to send an alarm signal with the alarm attribute to an external device, such as an alarm forwarding system, escalation management system, etc. The features described herein help prevent alarm fatigue and obscurity and can also help reduce patient discomfort and stress while receiving therapy with the infusion pump. These and other embodiments are described in greater detail below with reference to FIGS. 1-9.

In one implementation, an infusion pump is configured to execute one or more alarm actions according to an alarm protocol. The infusion pump includes a processor; and a memory in communication with the processor. The memory is configured to store instructions that when executed by the processor cause the execution of an alarm manager. The alarm manager is configured to: receive alarm configuration information comprising one or more alarm protocols, each alarm protocol comprising one or more alarm parameters; determine the presence of an error condition; and perform an alarm action associated with the one or more alarm parameters in response to determining the presence of the error condition.

Each alarm protocol may comprise one or more context parameters associated with each alarm parameter. The context parameters may comprise one or more of a drug identifier, an infusate identifier, a therapy identifier, a concentration, or a clinical care area. The alarm parameters may comprise one or more alarm limits, alarm types, alarm rules, or alarm behaviors. The alarm parameters may comprise one or more of an alarm priority, an alarm criticality, or alarm audio information.

The alarm protocol may define a period during which a visual alarm on the infusion pump is activated, and an audible alarm on the infusion pump is not activated. The the alarm protocol may define a period during which a visual alarm is activated, an audible alarm is not activated, and an alarm signal indicative of an alarm attribute corresponding to a priority level of the alarm attribute is transmitted to an alarm forwarding system. The alarm protocol may define a period during which a visual alarm is activated, an audible alarm is activated, and an alarm signal indicative of an alarm attribute corresponding to a priority level of the alarm attribute is transmitted to an alarm forwarding service.

The alarm manager may be further configured to receive the alarm configuration information from a drug library. The alarm manager may be further configured to escalate an alarm by changing an alarm attribute and communicating the alarm attribute to an alarm forwarding system.

In another implementation, a method of managing alarms of an infusion pump according to an alarm protocol, comprises: receiving alarm configuration information comprising one or more alarm protocols, each alarm protocol comprising one or more alarm parameters; determining the presence of an error condition; and performing an alarm action associated with the one or more alarm parameters in response to determining the presence of the error condition.

Each alarm protocol may comprise one or more context parameters associated with each alarm parameter. The context parameters may comprise one or more of a drug identifier, an infusate identifier, a therapy identifier, a concentration, or a clinical care area. The alarm parameters may comprise one or more alarm limits, alarm types, alarm rules, or alarm behaviors. The alarm parameters may comprise one or more of an alarm priority, an alarm criticality, or alarm audio information.

The method may further comprise, during a period, activating a visual alarm on the infusion pump and not activating an audible alarm on the infusion pump. The method may further comprise, during a period, activating a visual alarm on the infusion pump, not activating an audible alarm on the infusion pump, and transmitting an alarm signal indicative of an alarm attribute corresponding to a priority level of the alarm attribute to an alarm forwarding system. The method may further comprise, during a period, activating a visual alarm on the infusion pump, activating an audible alarm on the infusion pump, and transmitting an alarm signal indicative of an alarm attribute corresponding to a priority level of the alarm attribute to an alarm forwarding service.

Receiving the alarm configuration information may comprise receiving the alarm configuration information from a drug library. The method may further comprise escalating an alarm by changing an alarm attribute and communicating the alarm attribute to an alarm forwarding system.

### BRIEF DESCRIPTION OF THE DRAWINGS

The embodiments described herein are illustrated by way of example, and not by way of limitation, in the figures of the accompanying drawings in which like references indicate similar elements.

FIG. 1A is a schematic diagram of an example network environment including one or more networked infusion pumps in accordance with aspects of this disclosure.

FIG. 1B is a block diagram of another example network environment, including an example clinical environment and an example cloud environment, in accordance with aspects of this disclosure.

FIG. 1C is a block diagram illustrating components of an example cloud environment in accordance with aspects of the present disclosure.

FIG. 2 is a block diagram illustrating components of an example infusion pump having an alarm manager in accordance with aspects of the present disclosure.

FIG. 3 is a block diagram illustrating communication between an infusion pump and various components and individuals within a clinical environment.

FIG. 4 illustrates an example structure of a drug library that includes alarm configuration settings (e.g., alarm limit rules and alarm limits)

FIG. 5 is a block diagram of infusion pump determining an alarm therapy based upon a clinical therapy provided to a patient.

FIG. 6 illustrates an example block diagram of an alarm protocol.

FIG. 7 illustrates a method performed by an alarm manager to determine whether to activate various alarms.

FIG. 8 illustrates example protocols for activating one or more alarms.

FIG. 9 illustrates another method performed by an alarm manager to control the activation of various alarms.

### DETAILED DESCRIPTION

### Introduction

An infusion pump for infusing one or more fluids into a medical patient may be programmed by a user to infuse a particular drug according to various treatment parameters, such as dose, rate, volume, and/or duration of time. Certain clinical values may be entered into the infusion pump by the user, retrieved from a database (e.g., an electronic medical record (“EMR”), etc.) over a hospital network, and/or determined by the pump from one or more sensors (e.g., location). These clinical values may be used to determine one or more of the treatment parameters used to deliver a desired infusion therapy to the medical patient.

In addition, a drug library accessed by the infusion pump includes one or more rules that define predetermined safe ranges of values for the treatment parameters. For a given drug, the drug library may define lower and upper values of the treatment parameters that have been determined to be safe for use with the particular drug. The lower and upper values may be referred to as “soft limits.” The range of values between the lower and upper soft limits define a safe range of values of the treatment parameter of interest. For a given drug, the drug library may also define lower and upper values of each treatment parameters that may not be exceeded by the user under any circumstance. These lower and upper values may be referred to as “hard limits.” The pump will not allow the selection of a treatment parameter value that is below the lower hard limit or above the upper hard limit under any circumstance. Such rules are generally determined at least based upon the particular drug to be administered by the infusion pump. In addition, the rules for a given drug may vary based upon the concentration of the drug to be administered and/or the clinical care are of the hospital in which the drug is to be administered, and/or other drug library parameters.

The range of values between the soft and hard limits are values that may be selected by the user, but only after confirmation from the user that the user wishes to override the soft limit restriction. The pump keypad may provide the user with an override button for the user to provide such confirmation. Hard limit restrictions may not be overridden.

For example, a user may program a pump to deliver the drug dopamine (of a particular concentration, such as, for example, 400 mg/250 mL) to a medical patient. The pump may access a drug library and determine that the lower and upper hard limits for dosing this particular drug are 0 and 50 mcg/kg/min (micrograms of drug per kilogram of patient weight per minute). Therefore, the user will not be able to enter and submit a dosing value for this drug that is below 0 mcg/kg/min or above 50 mcg/kg/min The user will not be able to override the hard limit restriction. The drug library may also indicate that the lower and upper soft limits for dosing this particular drug are 2.5 mcg/kg/min and 20 mcg/kg/min. Therefore, the user will not be able to enter and submit a dosing value for this drug that is below 2.5 mcg/kg/min or above 20 mcg/kg/min unless the user activates an override key to override the soft limit restriction. The user may enter and submit a value within the range defined by the lower and upper soft limits (e.g., between 2.5 mcg/kg/min and 20 mcg/kg/min) without restriction.

In addition, the drug library may also include alarm configuration information that can be used by an alarm manager to determine one or more alarm protocols based at least in part upon a drug, infusate, and/or therapy delivered to the patient. For example, the alarm configuration information may indicate for a particular infusate and alarm condition, a first action may be performed by the pump during a first alarm period, a second action may be performed during a second alarm period, a third action may be performed by the pump during a third alarm period, and a fourth action may be performed during a fourth alarm period. Additional or fewer actions and/or alarm periods may be defined for a particular infusate.

For example, if the infusate is saline and the error condition is a detection of a distal occlusion error or the completion of a volume-to-be-infused infusion, the alarm protocol may define the following actions and periods: (1) during a first period (e.g., 5 min., 10 min , etc.), activate a visual alarm on the pump and monitor the error condition to see if it is resolved; (2) during a second period (e.g., a subsequent 5 min, 10 min., etc.) maintain the visual alarm on the pump, set an alarm attribute to LOW, and transmit an alarm signal indicative of the alarm attribute (e.g., send the alarm signal to an alarm forwarding service, a nurse's station, etc.); (3) during a third period (e.g., a subsequent 5 min., 10 min., etc.) maintain the visual alarm on the pump, activate an audible alarm on the pump, update the alarm attribute to MEDIUM, and transmit the alarm signal; (4) during a fourth period (e.g., a subsequent 5 min., 10 min., etc.) maintain the visual alarm on the pump, increase one or more parameters of the audible alarm (e.g., volume, tone, “beeping” frequency, etc.), update the alarm attribute to HIGH, and transmit the alarm signal.

The user may program other treatment parameters, e.g., infusion rate, volume-to-be-infused (“VTBI”), and/or infusion duration in a similar manner Selection of such treatment parameter values may be similarly restricted by lower and upper soft and hard limit values for each of such treatment parameters.

The drug library and/or the lower and upper soft and hard limit restrictions and/or the alarm configuration information may be determined not only based upon the drug to be infused, but also based upon one or more context parameters. Such context parameters include, for example, the drug to be infused, the drug concentration, and/or the clinical care area in which the pump is located. The drug library that is utilized and/or the lower and upper soft and hard limit restrictions and/or the alarm configuration information may vary based upon the any one or more of the context parameters, such as, for example, location of the infusion pump. If the infusion pump is located in a neonatal intensive care unit (NICU), for example, a drug library may be selected to provide smaller lower and upper soft and limit values for a drug and/or shorter alarm period settings for a particular alarm configuration than if the infusion pump were located at location in the hospital that provides treatment to grown adults.

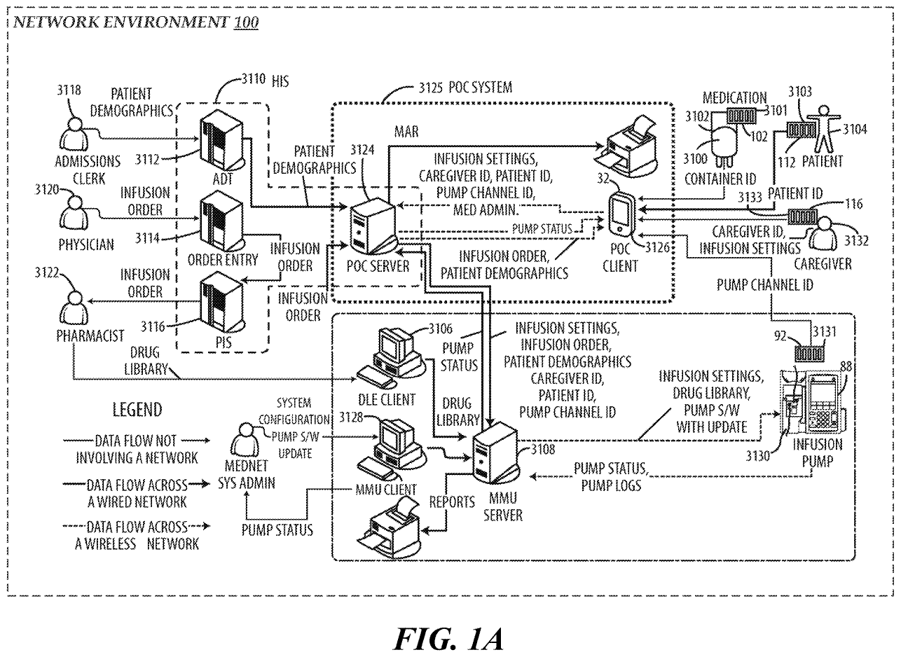

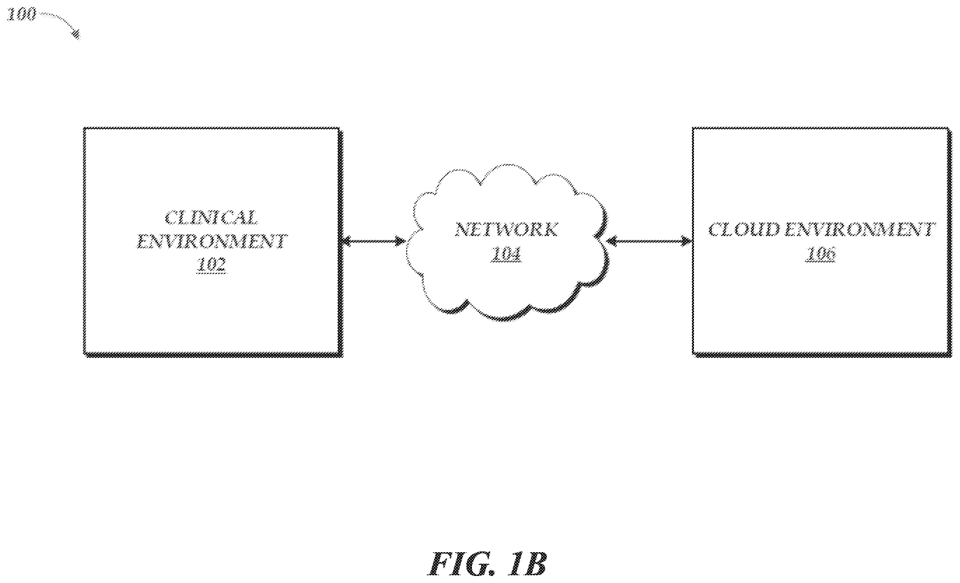

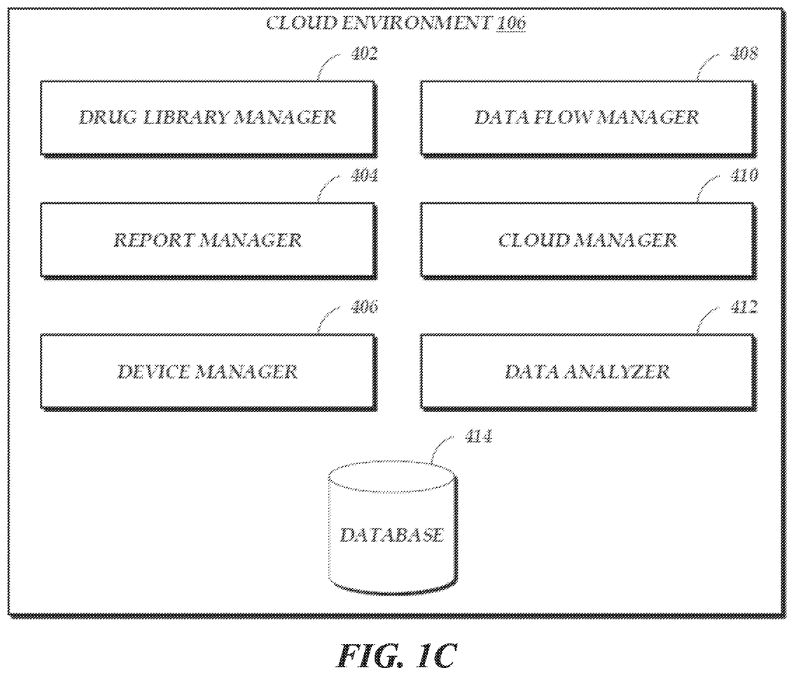

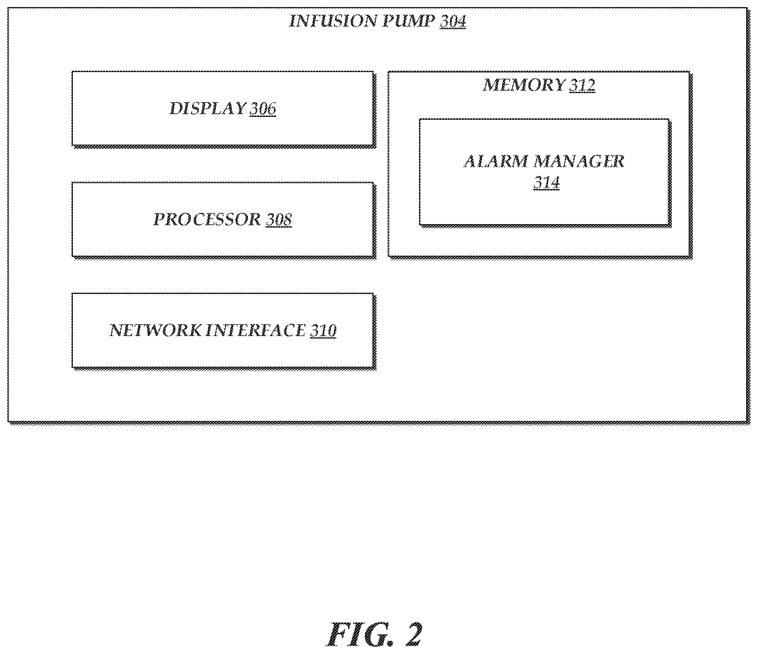

With reference to FIGS. 1A-1C, example network environments in which one or more infusion pumps implementing one of the techniques of the present disclosure may be utilized are described. Following the discussion of FIGS. 1A-1C, specific details of the various embodiments of the present disclosure are described with reference to FIGS. 2-9.

### Overview of Example Network Environment

FIG. 1A illustrates one embodiment of a system for administering medication via an infusion pump in a network environment 100. The medication management system (MMS) shown in FIG. 1A includes a medication management unit (MMU) server 3108 and a medical device, such as infusion pump 3130, operating in conjunction with one or more information systems or components of a hospital environment. The various components (e.g., systems, servers, computing devices, etc.) of the network environment 100 may be physically located within a single location (e.g., a hospital, clinic, etc.) or may be distributed over multiple locations. For example, the different components of the network environment 100 may communicate with each over the Internet and/or over one or more additional networks. One or more components of the network environment 100 may be located in the cloud, at a remote location from the hospital or clinical environment. For example, in some embodiments, the network environment 100 includes the network embodiment discussed below with respect to FIGS. 1B and 1C.

Intravenous (IV) fluid(s) and/or medication(s) 3100 in containers 3102 may be administered to a patient 3104 using the system shown in FIG. 1A. Although the system shown in FIG. 1 utilizes barcodes and a barcode reader as apparatus to input and read machine-readable information, those skilled in the art will appreciate that other apparatus for reading or inputting information may be utilized. Moreover, a point of care (POC) client 3126 may include an identification receiver 32 adapted to recognize such indicia may be provided in the MMS.

In certain aspects, the IV fluids and/or medications 3100 in container 3102 may be provided with new or supplemental labels with a unique infusion order identifying barcode by a pharmacist according to certain hospital practices. Specifically, drug container specific identification information, such as barcoded information on the container 3102 may include patient identification information, medication identification information, universal identification information, medical device delivery information, and/or medication order information. The IV fluids and/or medications 3100 in barcode-identified containers 3102 may be supplied to hospitals by various vendors, with preexisting unique barcode identifiers, which include medication information and other information, such as a National Disease Center (NDC) code, expiration information, drug interaction information, and the like.

In some aspects of the disclosure, the universal identification information on the container 3102 may be a unique medication order identifier that, by itself, identifies the order associated with the container. In other aspects, the identification information on the container 3102 may be a composite patient/order code that contains both a patient ID (such as a medical record number) and an order ID unique only within the context of the patient. In certain aspects, the identification information on the container 3102 may include a medication ID. The system identified in FIG. 1A may include a drug library editor (DLE) client 3106, such as a notebook, desktop or server computer. The DLE client 3106 may include DLE software. As described above, the MMU server 3108 may have MMU software that is installed and runs on the MMU server 3108. The drug library and other databases may be stored on the MMU server 3108, on a separate server, and/or in remote locations.

Hospital information systems (HIS) 3110 may include one or more computers connected by cabling, interfaces, and/or Ethernet connections. Alternatively, wireless connections and communications may be used in whole or in part. Servers provide processing capability and memory for storage of data and various application programs or modules, including but not limited to an admissions-discharge-and-transfer (ADT) module or computer 3112, a computerized physician order entry (CPOE) module or computer 3114, and a pharmacy information system (PIS) module or computer 3116. Hospital personnel, such as admission clerks 3118, physicians 3120, and pharmacists 3122, respectively, may be authorized to access these modules through client workstations connected to the servers in order to enter data, access information, run reports, and complete other tasks.

In the embodiment shown in FIG. IA, the HIS 3110 may also include a POC system 3125 including a server or POC computer 3124 (sometimes referred to as a barcode point of care server or computer), or the POC computer 3124 may be separate from the HIS 3110. The POC computer 3124 may act as a part of the POC system 3125 (sometimes referred to as the barcode point of care system or BPOC) and may be able to wirelessly communicate through a plurality of wireless communication nodes located throughout the hospital, utilizing a wireless communications protocol, such as IEEE 801.11, IEEE 802.11, or Bluetooth. The POC computer 3124 may communicate wirelessly with a portable thick client, POC client 3126, carried by a caregiver. The POC client 3126 may be a personal digital assistant (PDA) that includes significant memory, display, and processing capabilities. The POC client device may execute a variety of programs stored in its memory in some degree independently of the POC computer 3124.

In one embodiment of FIG. 1A, the MMU server 3108 may be hard-wired to the DLE client 3106 and to a MMU client 3128. Alternatively, the MMU and DLE client functions may be combined onto a single client computer/workstation or may reside together with the MMU server 3108 on a single combined MMU/DLE server. The MMU server 3108 may reside in a location remote from the patient's room or treatment area. For instance, the MMU server 3108 may reside in a secure, climate-controlled information technology room with other hospital servers, and computer equipment and its client terminals may be located in the pharmacy, biomedical engineering area, nurse station, or ward monitoring area. One MMU server 3108 may monitor, coordinate, and communicate with many infusion pumps 3130. For example, in one embodiment, the MMU software running on the MMU server 3108 may support up to one thousand infusion pumps concurrently.

In the embodiment of FIG. 1A, the POC client 3126 in the POC system 3125 may communicate through the POC server 3124 with the MMU server 3108. The MMU server 3108 may interface or communicate wirelessly with the infusion pump 3130 through the same wireless nodes utilized by the POC system 3125 and a connectivity engine and antenna on or in the infusion pump 3130. Communication between the infusion pump 3130 and the POC client 3126 may take place through the MMU server 3108 and POC server 3124. The MMU server 3108 may store in an associated memory both the logical ID and the network ID or Internet Protocol (IP) address of the infusion pump(s) 3130, such that only the MMU server 3108 may communicate in a direct wireless manner with the infusion pump 3130. Alternatively, the MMU server 3108 may provide the IP address and other information about the infusion pump 3130 to the POC system 3125 to facilitate direct communication between the POC system 3125 and the infusion pump 3130.

Upon admission to the hospital, the admission clerk 3118 or similar personnel may enter demographic information about each patient 3104 into an associated memory of the ADT module or computer 3112 of an HIS database stored in an associated memory of the HIS 3110. Each patient 3104 may be issued a patient identification wristband, bracelet, or tag 112 that may include an identifier 3103, such as a barcode or RFID tag, identifying the patient. The wristband, bracelet, or tag 112 may also include other information, in machine readable or human-readable form, such as the name of the patient's doctor, blood type, allergies, and the like.

The patient's doctor 3120 may prescribe medical treatment by entering a medication order into the CPOE module or computer 3114 within the HIS 3110. The medication order may specify a start time, stop time, a range of allowable doses, physiological targets, route, and site of administration. In the case of an order for infusion of fluids or medication, the order may be written in various formats, and may include the patient's name, patient ID number, a unique medication order or prescription number, a medication name, medication concentration, a dose or dosage, frequency, and/or a time of desired delivery. This information may be entered into the memory of the CPOE module or computer 3114 and may be stored in a memory associated with at least the POC server 3124.

The medication order may also be delivered electronically to the PIS module or computer 3116 in the pharmacy and may be stored in an associated memory. The pharmacist 3122 may screen the prescribed order, translate it into an order for dispensing medication, and prepare the medication or fluids with the proper additives and/or necessary diluents. The pharmacist 3122 may prepare and affix a label 102 with drug container specific identifying information 3101 to the medication or drug container 3102. The label may include in machine-readable and/or human-readable form medical device specific delivery information including but not limited to the dispense ID number, patient ID, drug name, drug concentration, container volume, volume-to-be-infused (“VTBI”), rate, duration, and the like. Only two of the three variables VTBI, rate, and duration may be defined as the third may be calculated when the other two are known. The labeled medication may be delivered to a secure, designated staging location or mobile drug cart on the ward or floor near the patient's room or treatment area. The medication order pending dispensing or administration may be posted to a task list in the HIS 3110 and POC system 3125 and stored in an associated memory.

The caregiver 3132 (e.g., a nurse) may use the identification receiver 32 associated with the POC client 3126 to scan his/her caregiver identification badge 116 and enter a password, which logs the caregiver into the system and authorizes the caregiver to access a nurse's task list from the POC system 3125 through the POC client 3126. The caregiver 3132 may view from the task list that IV drugs are to be administered to certain patients 3104 in certain rooms. The caregiver 3132 obtains the necessary supplies, including medications, from the pharmacy and/or a staging area in the vicinity of the patient's room.

The caregiver 3132 may take the supplies to a patient's bedside, turn on the infusion pump 3130, verify that the network connection icon on the infusion pump 3130 indicates a network connection (for example, a wireless connection such as Wi-Fi or the like) is present, select the appropriate clinical care area (CCA) on the infusion pump 3130, and mount the IV bag, container, or vial 3102 and any associated tube set as required in position relative to the patient 3104 and infusion pump 3130 for infusion. Another connection icon on the infusion pump 3130 or pump user interface screen can indicate that a wired or wireless connection to the MMU server 3108 is present. Using the identification receiver/reader integral to the POC client 3126, the caregiver 3132 may scan the barcode on the patient's identification wristband, bracelet, or tag 112 or other patient identification device. A task list associated with that particular patient may appear on the POC client 3126 screen. The task list, which may also include orders to give other forms of treatment or medication by other routes (oral, topical, etc.), may be obtained from the HIS 3110 via the POC server 3124 and communicated wirelessly to the POC client 3126. In one embodiment, the list is generated by matching the scanned patient ID with the patient ID for orders in memory within the POC server 3124. In another embodiment, the order information may be obtained by scanning the drug container specific identification information for associated orders in memory within the POC server 3124, through the following step(s).

The caregiver 3132 may scan the medication barcode label 102 containing medication container specific identification information 3101 on the medication container 3102 with the POC client 3126. The POC client 3126 may highlight the IV administration task on the task list and send the scanned medication container specific identification information, such as dispense ID information, from the medication container 3102, to the POC server 3124. The POC server may use the medication container specific identification information to pull together the rest of the order details and send them back to the POC client 3126. The POC client 3126 may then display an IV Documentation Form on its screen. One side of the IV Documentation Form screen may show the order details as “ordered” and the other side may be reserved for a status report from the infusion pump 3130. The status report from the infusion pump 3130 may be transmitted to the POC client 3126 through the POC server 3124 and MMU server 3108. The lower portion of the IV Documentation Form screen may provide the caregiver 3132 with instructions (like to scan the infusion pump 3130 barcode) or identify whether the pump is running or stopped.

The caregiver 3132 may then scan the barcode label 92 associated with the infusion pump 3130 (or pump channel if the pump is a multi-channel pump). The barcode label 92 may contain medical device specific identification information 3131, such as the logical name and/or logical address of the device or channel. The POC system 3125 then automatically bundles the information into a program pump request containing the “order details” and in one embodiment, without further interaction with the caregiver 3132, transmits this information to the MMU server 3108.

The program pump request may include at least some of the following information (in HIS/POC system format): a Transaction ID, which may include a Logical Pump ID, a Pump Compartment, a Pump Channel ID, a Reference Device Address, a Caregiver ID, a Caregiver Name, a Patient/Person ID (HIS identifier), a Patient Name, a Patient Birth Date & Time, a Patient Gender, a Patient Weight, a Patient Height, and an Encounter ID which may include a Room, a Bed, and a Building (including CCA). The program pump request may also include Order Information or “order details”, including an Order ID, a Start Date/Time, a Stop Date/Time, a Route of Administration, a Rate, a Duration of Infusion (Infuse Over), a Total Volume to be Infused (VTBI), an Ad Hoc Order Indicator, and Ingredients including HIS Drug Name or HIS Generic Drug Name, HIS Drug Identifier or HIS Generic Drug ID, Rx Type (Additive or Base), Strength w/units, and Volume w/units. The program pump request may further include Patient Controlled Analgesia (PCA) Orders Only information, such a PCA Mode-PCA only, Continuous only, or PCA and Continuous, a Lockout Interval (in minutes), a PCA Continuous Rate, a PCA Dose, a Loading Dose, a Dose Limit, a Dose Limit Time w/ units, a Total Volume in vial or syringe, and Order Comments.

The MMU server 3108 may map or convert the wide range of expressions of units allowed by the HIS 3110 or POC system 3125 for POC client 3126 requests into the much more limited set of units allowed in the MMU server 3108 and infusion pump 3130. For example, the POC client 3126 request may express “g, gm, gram, or grams” whereas the MMU server 3108 and/or infusion pump 3130 may accept “grams” only. Infusion pump 3130 delivery parameters or infusion pump 3130 settings are mapped or converted from corresponding order information or “order details” of the program pump request.

The MMU server 3108 may store in an associated memory a mapping or translation table that keep track of the logical ID, serial number or other identifier of an infusion pump 3130 and the corresponding current network (static or dynamic) address (Internet Protocol (IP) address) or ID of the infusion pump 3130 on the network, which in this example is a wireless network. The MMU server 3108 may be able to translate or associate a given identifier of the infusion pump 3130 with its network address in the translation table and provide the network IP address to the requesting POC system 3125 or device. The MMU server 3108 may also store in an associated memory and/or look up the drug library applicable to the scanned infusion pump 3130 and/or convert the Drug ID and Strength from the pump program request into an index number of the medication at the desired strength or concentration from the drug library. The duration of the infusion may come from the POC system 3125 in hours and minutes and may be converted to just minutes for the infusion pump 3130 to recognize it. Volume or VTBI may be rounded to provide a value-specific and infuser-specific number of digits to the right of the decimal point. Units (of drug) may be converted to million units where appropriate. Patient weight may be converted and either rounded according to infuser-specific rules or not sent to the infuser.

Once the MMU server 3108 transforms the information from the program pump request into infusion pump settings or delivery parameters and other information in a format acceptable to the infusion pump 3130, the MMU server 3108 may wirelessly download a command message to the infusion pump 3130. If the infusion pump 3130 is not already equipped with the latest appropriate version of the hospital-established drug library, the MMU server 3108 may also automatically download a drug library to the infusion pump 3130. The hospital-established drug library may be maintained in a separate process undertaken by the biomedical engineer or pharmacist 3122 to place limits on the programming of the infusion pump 3130, as well as other infusion pump operating parameters such as default alarm settings for air in the line, occlusion pressure, and the like. The drug library may set up acceptable ranges or hard and/or soft limits for various drug delivery parameters in the infusion pump 3130.

The MMU server 3108 may also download to the infusion pump new versions, patches, or software updates of the infusion pump's internal operating system software. The infusion settings or delivery parameters and other information from the MMU server 3108 may be entered into the memory of the infusion pump 3130 and the infusion pump 3130 settings may automatically populate the programming screen(s) of the infusion pump 3130, just as if the caregiver 3132 had entered the information and settings manually. The infusion pump 3130 screen may populate with the name of the drug and drug concentration based on the drug library index number, patient weight, rate, VTBI, and/or duration. Further, the MMU server 3108 may transmit one or more synchronization signals or screen content display rules/parameters to the infusion pump 3130. A return message of confirmation signal may be sent to the MMU server 3108 by the infusion pump 3130 to indicate that the command message has been received. At this point, if necessary, the caregiver 3104 may manually enter any additional infusion settings or optional information that was not included in the command message.

The infusion pump 3130 may then prompt the caregiver 3132 to start the infusion pump 3130 by pressing the start button. When the caregiver 3132 presses the start button, a confirmation screen with the infusion settings programmed may be presented for confirmation and an auto-program acknowledgment message can be sent to the MMU server 3108 to forward without request (e.g., pushed in a near real-time manner) or provide to the POC system 3125 when requested or polled. When the caregiver 3132 presses the button to confirm, the infusion pump 3130 may begin delivering fluid according to the programmed settings. The infusion pump 3130 may send a status message to the MMU server 3108 indicating that the infusion pump 3130 was successfully auto-programmed, confirmed and started by the caregiver 3132, and is now delivering fluid. This information may also be displayed at the infusion pump. The MMU server 3108 may continue to receive logs and status messages wirelessly from the infusion pump 3130 periodically as the infusion progresses or when alarms occur.

The MMU server 3108 may report a portion of the initial status message to the POC client 3126 through the POC server 3124 (in MMU format) to indicate that the infusion pump 3130 has been auto-programmed and the caregiver 3132 has confirmed the settings. The MMU server 3108 may communicate to the POC system 3125 and/or at the infusion pump 3130 the actual Rate, VTBI, and Duration. A notation at the bottom of the screen of the POC client and/or the infusion pump may indicate that the infusion pump 3130 is running. The infusion pump 3130 may compare and give a visual, audio, or other type of affirmative signal if the pump information matches or acceptably corresponds with the ordered information. An initial determination of whether the pump information matches the order may be done in the MMU server 3108 and communicated to the POC client 3126 through the POC server 3124. Alternatively, the POC server 3124 or the infusion pump 3130 may make the necessary comparisons. If the pump information does not match the order, the infusion pump 3130 at the display 88 may output a visual, audio, or other type of negative signal, which may include an error message.

The caregiver 3132 may be prompted to review and press a save button on the infusion pump 3130 if the order has been begun as desired or any variations are acceptable. The MMU server 3108 may receive status, event, differences, and variation information from the infusion pump 3130 and pass such information to the POC system 3125. In a separate subsequent step, the nurse may electronically sign the record and presses a send button on the POC client POC client 3126 to send the information to the patient's electronic medication record (EMR) or medication administration record (MAR).

### Additional Example Network Environment

FIG. 1B illustrates a network environment 100 in which a clinical environment 102 communicates with a cloud environment 106 via a network 104. The clinical environment 102 may include one or more healthcare facilities (e.g., hospitals). The network 104 may be any wired network, wireless network, or combination thereof. In addition, the network 104 may be a personal area network, local area network, wide area network, over-the-air broadcast network (e.g., for radio or television), cable network, satellite network, cellular telephone network, or combination thereof. For example, the network 104 may be a publicly accessible network of linked networks such as the Internet. For example, the clinical environment 102 and the cloud environment 106 may each be implemented on one or more wired and/or wireless private networks, and the network 104 may be a public network (e.g., the Internet) via which the clinical environment 102 and the cloud environment 106 communicate with each other. The cloud environment 106 may be a cloud-based platform configured to communicate with multiple clinical environments. The cloud environment 106 may include a collection of services, which are delivered via the network 104 as web services. The components of the cloud environment 106 are described in greater detail below with reference to FIG. 1C.

### Components of Clinical Environment

The clinical environment 102 may include one or more clinical IT systems, one or more infusion pumps, and optionally, one or more connectivity adapters. Further, the clinical environment 102 may be configured to provide cloud user interfaces (e.g., generated and provided by the cloud environment 106). The clinical IT system may include a hospital infusion system (HIS) designed to manage the facilities' operation, such as medical, administrative, financial, and legal issues and the corresponding processing of services. The HIS can include one or more electronic medical record (EMR) or electronic health record (EHR) systems, as well. The infusion pump is a medical device configured to deliver medication to a patient. The connectivity adapter is a network component configured to communicate with other components of the clinical environment 102 and also communicate with the cloud environment 106 on behalf of the other components of the clinical environment 102. In one embodiment, all messages communicated between the clinical environment 102 and the cloud environment 106 pass through the connectivity adapter. In some cases, the connectivity adapter is a network appliance with limited storage space (e.g., memory and/or persistent storage). The cloud user interfaces may be provided to a user in the clinical environment 102 via a browser application, desktop application, mobile application, and the like. The user may access status reports and other data stored in the cloud environment 106 via the cloud user interfaces.

The components of the clinical environment 102 may communicate with one or more of the other components in the clinical environment 102. For example, each of the clinical IT system and the infusion pump may communicate with the connectivity adapter via physical local area network (LAN) and/or virtual LAN (VLAN). The clinical environment 102 may include other medical devices and non-medical devices that facilitate the operation of the clinical environment 102.

### Components of Cloud Environment

FIG. 1C illustrates one embodiment of a cloud environment 102, which includes can include one or more of a drug library manager (DLM) 402, report manager 404, device manager 406, data flow manager (DFM) 408, cloud manager (CM) 410, data analyzer (DA) 412, and database 414.

The DLM 402 may provide a set of features and functions involved in the creation and management of drug libraries for use with infusion pumps. These drug libraries may provide user-defined settings for pump configuration and drug infusion error reduction.

The report manager 404 may provide various reporting capabilities for clinically relevant infusion data which users can choose to use for further analysis, such as tracking and trending of clinical practices.

The device manager 406 may oversee and manage the maintenance of infusion pumps, providing users the capability to view and manage asset and operational data. For example, the device manager 406 may schedule drug library and software updates for infusion pumps.

The DFM 408 may facilitate storing, caching, and routing of data between compatible infusion pumps 204, connectivity adapters 206, cloud services (e.g., infusion pump management software including modules 402-414 of FIG. 1C), and external systems. For example, the DFM may store infusion and operational data received from infusion pumps, store and cache infusion pump drug libraries and software images, convert and route network messaging between the cloud environment 106 and the clinical environment 102, convert and route medication order information from a hospital information system to an infusion pump (e.g., auto-programming or smart-pump programming), and/or convert and route alert information and infusion events from infusion pumps to hospital information systems (e.g., alarm/alert forwarding, and auto-documentation, or infusion documentation).

The CM 410 may serve as a computing platform for the other modules illustrated in FIG. 1C. Functionally, the CM 410 may be similar to Microsoft Windows® or Linux® operating systems as it provides the following services: networking, computation, user administration and security, storage, and monitoring.

The DA 412 may provide data analytics tools for generating user interfaces and reports based on the data generated and/or received by the other modules illustrated in FIG. 1C.

The database 414 may store data generated and/or received by the modules 402-412 of the cloud environment 106. Although not illustrated in FIG. 1C, the cloud environment may provide other resources such as processors, memory, disk space, network, etc. The modules 402-412 may be hardware components configured to perform one or more of the techniques described herein. Alternatively, the modules 402-412 may be implemented using software instructions stored in physical storage and executed by one or more processors. Although illustrated as separate components, the modules 402-412 may be implemented as one or more hardware components (e.g., a single component, individual components, or any number of components), one or more software components (e.g., a single component, individual components, or any number of components), or any combination thereof.

In some embodiments, the cloud environment 106 can be implemented using a commercial cloud services provider (e.g., Amazon Web Services®, Microsoft Azure®, Google Cloud®, and the like). In other embodiments, the cloud environment can be implemented using network infrastructure managed by the provider and/or developer of the modules 402-412 shown in FIG. 1C. In some embodiments, the features and services provided by one of more of the modules 402-412 may be implemented on one or more hardware computing devices as web services consumable via one or more communication networks. In further embodiments, one of more of the modules 402-412 are provided by one or more virtual machines implemented in a hosted computing environment. The hosted computing environment may include one or more rapidly provisioned and released computing resources, such as computing devices, networking devices, and/or storage devices.

### Other Environments

FIGS. 1A-1C illustrate example environments in which the various techniques of the present disclosure may be utilized. However, the embodiments described herein are not limited to such an environment and may be applied to any networked or non-networked environment. An example infusion pump that may be used in one or more of such environments is described below with reference to FIG. 2.

### Architecture of Infusion Pump

With reference to FIG. 2, the components of an example infusion pump are described in greater detail. The example architecture of the infusion pump 304 depicted in FIG. 2 includes an arrangement of computer hardware and software modules that may be used to implement aspects of the present disclosure. The infusion pump 304 may include many more (or fewer) elements and/or sub-elements than those shown in FIG. 2. It is not necessary, however, that all of these elements be shown in order to provide an enabling disclosure.

As illustrated, the infusion 304 includes a display 306, a processor 308, a network interface 310, and a memory 312, all of which may communicate with one another by way of a communication bus. The display 306 may display information generated or stored by the infusion pump 304 or any other information associated with the infusion pump 304. For example, infusion pump 304 may be used to deliver medication to a patient. In such a case, the display 306 may display the volume of the medication infused so far, the volume of the medication to be infused, the rate at which the medication is being infused, and the like. The display 306 may also provide a keypad to the user for data entry and programming.

The processor 308 may receive information and instructions from other computing systems or services via a network. The processor 308 may also transmit information to and receive information from the memory 312 and further provide content to the display 306 for display. The network interface 310 may provide connectivity to one or more networks or computing systems in the network environment described herein. For example, the network interface 310 may be a serial port, a parallel port, or any other communication interface that can enable or facilitate wired or wireless communication according to any communication protocols such as Zigbee (e.g., IEEE 802.15.4), Bluetooth, Wi-Fi (e.g., IEEE 802.11), Near Field Communication (NFC), and the like.

The memory 312 may contain computer program instructions (grouped as modules in some embodiments) that the processor 308 can execute in order to implement one or more aspects of the present disclosure. The memory 312 may include RAM, ROM, and/or other persistent, auxiliary, or non-transitory computer-readable media. In some embodiments, the memory 312 stores an operating system that provides computer program instructions for use by the processor 308 in the general administration and operation of the infusion pump 304. As illustrated in FIG. 2, the memory 312 may include an alarm manager 314. In some embodiments, the alarm manager 314 implements various aspects of the present disclosure.

Although not shown in FIG. 2, the infusion pump 304 may further include one or more input devices such as a touch screen, mechanical buttons, or a voice recognition system. Further, the infusion pump 304 may include one or more additional storage devices for storing data generated by the infusion pump 304 or other data utilized in implementing aspects of the present disclosure.

### Infusion Pump Alarm Manager Operating Environment

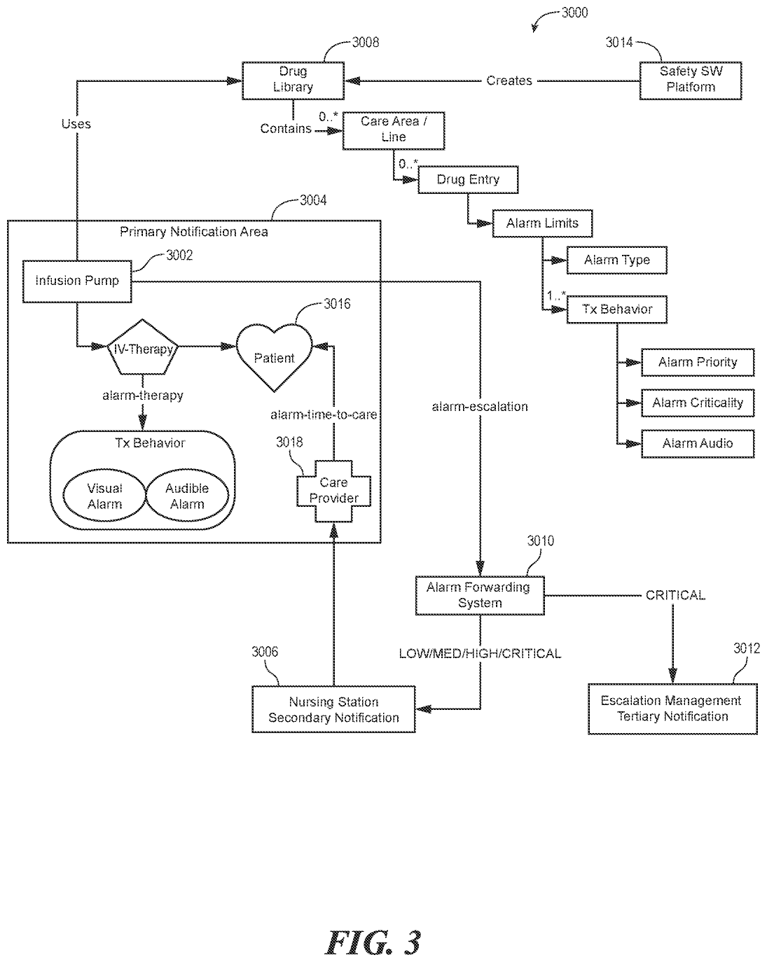

With reference now to FIG. 3, an operating environment 3000 for an infusion pump 3002 with an alarm manager includes primary 3004 and secondary 3006 notification areas (e.g., a nursing station, etc.), as well as a drug library 3008 and alarm forwarding system (“AFS”) 3010. The AFS 3010 communicates with one or more secondary notification areas 3006, such as for example, a nursing station. The AFS 3010 also communicates with an escalation management and/or tertiary notification system 3012. The nursing station is typically a centralized area in a hospital where nurses can monitor the status of multiple patients on the hospital floor or ward under the nurses' supervision. The escalation management and/or tertiary notification system 3012 is configured to send alarms, messages, and/or notifications to remote clinicians. For example, such systems 3012 can send text messages to mobile telephones and/or page a user on his or her pager. The AFS 3010 can be configured to take a particular action based upon an alarm attribute received from an infusion pump 3002. For example, the AFS 3010 can determine that regardless of the alarm attribute, a notification is sent to the nursing station 3006, but only CRITICAL alarms having CRITICAL alarm attributes are sent to the escalation management system 3012. In some embodiments, the operating environment does not utilize an AFS 3010, and instead the infusion pump 3002 communicates directly with the secondary notification (e.g., nursing station) 3006, escalation management, and/or tertiary notification systems 3012.

The operating environment 3000 also includes a drug library 3008, which can be configured by an operator using a safety software platform 3014. The drug library 3008 can include various alarm parameters (e.g., alarm limits, alarm types, and alarm transmission rules/behaviors, which can include alarm priority, alarm criticality, and alarm audio information). The alarm limits may be defined for a particular drug entry, and/or for a particular clinical care area or line. For example, the same drug could have different alarm limit information for different clinical care areas within a hospital.

The infusion pump 3002 resides within a primary notification area 3004, which also includes the patient 3016, and a care provider (e.g., nurse, doctor, etc.) 3018. The infusion pump 3002 is configured to deliver a particular infusion therapy based upon therapy information received by the pump 3002 (e.g., from the care provider, or over a network, e.g., from an electronic medical record associated with the patient). The infusion pump 3002 can determine an alarm therapy, or protocol that defines when various visual and/or audible alarms are to be activated based upon a particular error condition and alarm limits received from the drug library 3008.

The following non-limiting examples illustrate an alarm manager utilizing alarm configuration information to define various alarm protocols based upon a detected error condition and infusion therapy.

### Septic Patient—Central Venous Lines Jugular (High Risk Scenario, Important to Maintain Infusion)

Therapy—A septic patient is given a large volume IV Saline:

Error condition detected—VTBI Complete is triggered (Unique Error ID: 1a).

Alarm Protocol:
First alarm period (e.g., from time t0 to time t1). Protocol ID: 1a-t0—Send MED urgency alarm to AFS (alarm attribute set to MEDIUM)/VISUAL ALARM_ACTIVE (activate visual alarm on display)/AUDIBLE ALARM_SILENT (do not activate audible alarm).
If the primary-care team does not respond and address the alarm prior to the expiration of the first alarm period (from t0 to t1) then continue to second alarm period.
Begin second alarm period (e.g., from time t1 to time t2). Protocol ID: 1a-t1—Send HIGH urgency alarm to AFS (alarm attribute set to HIGH)/VISUAL ALARM_ACTIVE (maintain or update visual alarm on displays)/ACTIVATE AUDIBLE ALARM (activate audible alarm on infusion pump).
If the primary-care team does not respond and address the alarm prior to the expiration of the second alarm period (from t1 to t2) then continue to third alarm period.
Begin third alarm period (from time t2 to time t3). Protocol ID: 1a-t2—send ESCALATION urgency alarm (set alarm attribute to CRITICAL and send or cause AFS to send alarm signal indicative of alarm to alarm escalation manager)/VISUAL ALARM_ACTIVE (on infusion pump displays)/AUDIBLE ALARM_ACTIVE (activate or update audible alarm).
During this third alarm period the alarm manager escalates the alarm to a secondary-care-team (either directly or via an AFS).
Protocol ID: 1a-t3—At time t3, the alarm is cleared when the alarm error addressed by clinician.
When clinician silences the alarms, ALARM_ACTIVE/ALARM_SILENCED (no audible alarm).

A different error condition may be detected. For example, Distal Occlusion error condition is detected (Unique Error ID: 1b).

Alarm Protocol:
First time alarm period (time t0 to time t1). Protocol ID: 1b-t0—set alarm attribute to HIGH urgency alarm/show at the pump and activate audible alarm.
Second time period (time t1 to time t2). Protocol ID: 1b-t1—Activate ESCALATION urgency alarm/show at the pump and activate audible alarm.
The alarm manager escalates the alarm to a secondary-care-team (e.g., directly or via an AFS).
1b- at time t2—Alarm-Cleared when addressed by clinician.

### Surgery Recovery—Cervical Artificial Disc Replacement—Peripheral Intravenous Lines Hand (Low Risk Scenario—Less Important to Maintain Infusion)

A patient in recovery, IV saline:

Error condition: VTBI complete is triggered at pump (“Unique Error ID: 1c”).

Alarm Protocol:
During first alarm period (time t0 to time t1): Protocol ID: 1c-t0—Set alarm attribute to LOW and send LOW urgency alarm/show visually at the pump (but don't activate audible alarm).
Protocol ID: 1c-t1—Alarm-Cleared (e.g., cleared by a clinician)/Addressed (e.g., alarm condition self-corrects, such as by patient moving his hand to un-kink the infusion line/tubing).

Alarm Condition: Distal Occlusion is triggered (Unique Error ID: 1d).

Alarm Protocol:
First alarm period (time t0 to time t1). Protocol ID: 1d-t0—Set alarm attribute to LOW and send LOW urgency alarm/show visually at the pump (but don't activate audible alarm).
Provide sufficient time to see if the Distal Occlusion is self-corrected, if not proceed to second alarm period.
Second alarm period (time t1 to time t2). Protocol ID: 1d-t1—Set alarm attribute to MEDIUM and send MED urgency alarm/show visually at the pump and activate audible alarm.
If the primary-care-team/clinician does not respond and clear the alarm and address the alarm condition prior to the expiration of second alarm period, continue to the third alarm period.
Third alarm period (time t2 to time t3). Protocol ID: 1d-t2—set alarm attribute to HIGH and send HIGH urgency alarm/show visually at the pump and activate audible alarm.
If the primary-care-team/clinician does not respond and clear the alarm and address the alarm condition prior to the expiration of the third alarm period, continue to the fourth alarm period.
Fourth alarm period (time t3 to time t4). Protocol ID: 1d-t3—Set alarm attribute to CRITICAL and send CRITICAL urgency alarm (e.g., to AFS and/or to an escalation manager)/show visually at the pump and activate audible alarm.
At this point, the alarm manager escalates the alarm to a secondary-care-team (e.g., directly or via an AFS).
Protocol ID: 1d-t4—Alarm-Cleared when addressed by clinician.

### Alarm Therapy

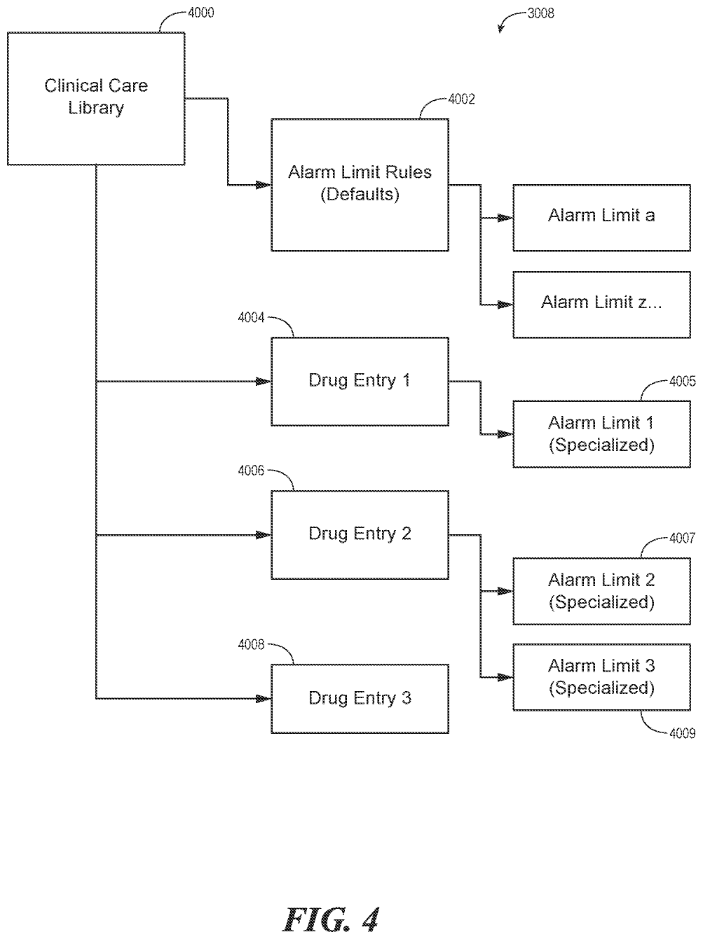

With reference now to FIG. 4, an example portion of a clinical care library portion 4000 of a drug library 3008 is illustrated. The clinical care library 4000 includes alarm limit rules 4002 that are default settings for a particular clinical care are, as well as alarm limits 4005, 4007, 4009 that are specific for particular drugs 4004, 4006, 4008 when delivered within the particular clinical care area. The alarm limit rules can define error conditions and alarm protocols, as discussed above. In addition to utilizing built-in alarm rules (e.g., visual, auditory, forwarding, priority, criticality, silencing rules, etc.) particular to a particular infusion pump (e.g., pump may have a default setting to activate a visual and audible alarm when an error condition is detected regardless of the therapy being delivered), the infusion pump according to various embodiments may utilize the alarm limit rules from a drug library 3008 to determine alarm protocols based upon an infusion therapy as well as a detected alarm condition. An alarm manager provides the infusion drug library 3008 a means to define specific behaviors based on therapy context. FIG. 4 illustrates one embodiment, such as creating specific alarm limit behaviors associated with drug entries within care containers/libraries (e.g., Clinical Care Area (CCA): ICU-Ped, etc.) or alarm behaviors for a care area.

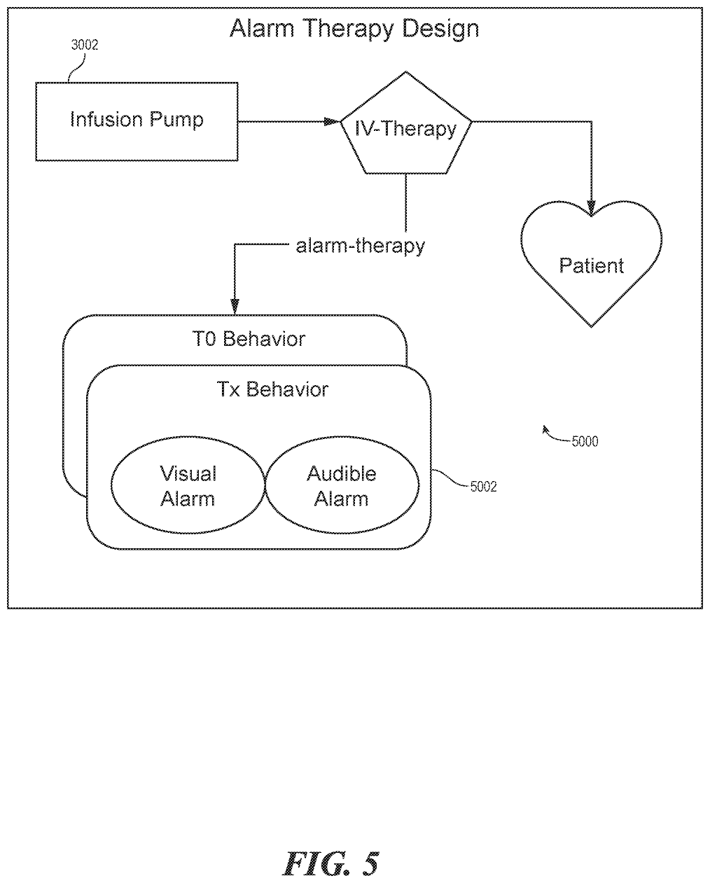

FIG. 5 illustrates one embodiment of an alarm manager protocol 5000, showing that alarm therapy, or an alarm protocol, may have different behaviors during different time periods based upon the therapy delivered by the infusion pump. For example, during different alarm periods (e.g., T0, T1, T2, . . . , Tx) 5002, each beginning at a different time (e.g., t0, t1, t2, . . . , tx), the alarm manager may cause different alarm actions, or behaviors, to occur. For example, the alarm action can include any of the actions described above, including, but not limited to, activate a visual alarm on the pump, activate an audible alarm on the pump, set an alarm attribute, and/or send an alarm signal indicative of the alarm attribute (e.g., to an AFS, escalation manager, nurse's station, etc.).

### Alarm Escalation

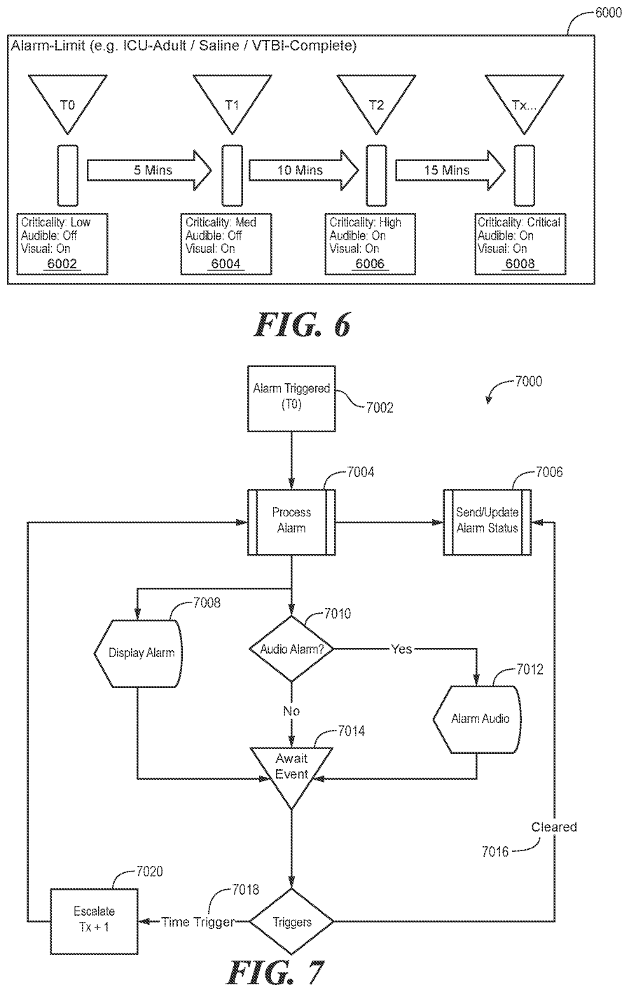

FIG. 6 illustrates an alarm escalation protocol 6000 according to one embodiment. The alarm escalation protocol 6000 can define alarm behavior (audible/priority/criticality, etc.) for one or more alarm time periods 6002. The alarm behavior may also be defined based upon the therapy being delivered by the infusion pump, as well. The alarm manager determines behavior, including whether escalation should occur, behavior based on time and care-protocol. This allows care providers an opportunity to prioritize care and provide a safety mechanism to escalate to tertiary notification systems. Alarms can be configured with only T0 (at the time of the event) or with multiple Tx escalations.

In the example protocol of FIG. 6, a first alarm period 6002 has a duration of 5 minutes (from t0 to t1), and during this first alarm period, the alarm attributes include: criticality=low, audible=off, and visual=on; a second alarm period 6004 (from t1 to t2) has a duration of 10 minutes, and during this second alarm period, the alarm attributes include: criticality=medium, audible=off, and visual on; a third alarm period 6006 (from t2 to t3 or tx) has a duration of 15 minutes, and during this third alarm period the alarm attributes include: criticality=high, audible=on, and visual=on; and during a final alarm period 6008 (beginning at t3 or tx), the alarm attributes include: criticality=critical, audible=on, and visual=on.

FIG. 7 illustrates a flow chart of one embodiment of an alarm escalation method 7000 that may be performed by the alarm manager According to the method 7000, an alarm (or error condition) is detected or triggered (e.g., at T0) 7002. The alarm manager processes the alarm 7004 according to an alarm protocol defined for the particular error condition detected and the alarm configuration data received from the drug library database. The alarm manager may send or update alarm status information 7006, e.g., to an AFS or other service as discussed herein. The alarm manager may also activate a visual indication of the alarm condition on the infusion pump 7008 and determine whether an audible alarm should be activated 7010. If yes, the alarm manager activates the alarm 7012. If not, the alarm awaits an event 7014 e.g., for a predetermined period of time (alarm period). The event could be, for example, that the error condition has self-corrected during the predetermined period of time. If the error condition has self-corrected 7016, such a “trigger” can result in the alarm being cleared by the alarm manager, the alarm status being updated, and the updated alarm status being send to the AFS. If the condition is not self-corrected, and the predetermined period of time (alarm period) ends 7018, such trigger can cause the alarm to escalate 7020 as a subsequent alarm period is entered. The escalated alarm status can be processed by the alarm manager 7004 according to the alarm protocol for the second time period. The method 7000 can repeat, entering subsequent time periods according to the alarm protocol, until the error condition is addressed, and the alarm is cleared.

### Alarm Time-to-Care

Alarm time-to-care refers to the alarm manager being configured to avoid disturbing the patient with stressful audible alarms until necessary. The alarm time-to-care feature adapts alarm behavior based upon therapy by providing various time windows or alarm periods during which the clinician is able to address error conditions or by allowing the error conditions to self-correct.

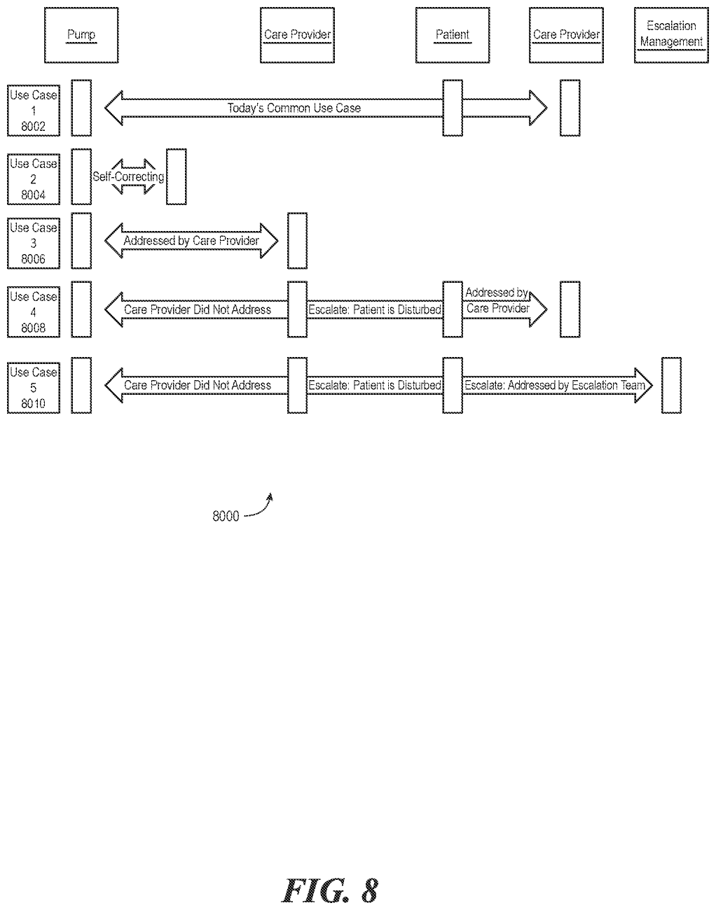

FIG. 8 illustrates five embodiments or use cases 8000 of an alarm protocol that defines one or more alarm periods during which a clinician is able to address error conditions or the infusion pump is monitored to determine if the error conditions self-correct. In the first embodiment (use case 1) 8002 an error condition is detected at the pump, alarms are activated at the pump, and remain active until they are addressed by a care provider. In the second embodiment (use case 2) 8004, an error condition is detected at the pump, and no alarm is activated on the pump (except optionally a visual alarm), and the error condition self corrects prior to any alarm activation. In the third embodiment (use case 3) 8006, an error condition is detected at the pump, no alarms are activated on the pump (except optionally a visual alarm), and the error condition is addressed by a care provider prior to audible activation at the pump. During the second and third embodiments 8004, 8006, the patient is not disturbed by the activation of an audible alarm at the infusion pump because the alarm is cleared before an audible alarm is sounded.

In the fourth embodiment (use case 4) 8008, an error condition is detected at the pump, during a first alarm period no alarms are activated on the pump (except optionally a visual alarm); however, the error condition is not addressed by a care provider. Therefore, the alarm manager proceeds to a second alarm period during which an audible alarm is activated and the patient is disturbed. The error condition is not addressed by a care provider during the second alarm period; therefore, the alarm manger proceeds to a third alarm period. The error condition is addressed by the care provider during the third alarm period. In the fifth embodiment (use case 5) 8010, an error condition is detected at the pump. During a first alarm period no alarms are activated on the pump (except optionally a visual alarm); however, the error condition is not addressed by a care provider. Therefore, the alarm manager proceeds to a second alarm period during which an audible alarm is activated and the patient is disturbed. The error condition is not addressed by a care provider during the second alarm period; therefore, the alarm manger proceeds to a third alarm period. The alarm manager causes the alarm to be escalated to an escalation team (e.g., send to an escalation manager directly or via an AFS). The error condition is addressed by the care provider during the third alarm period.

### Alarm Manager

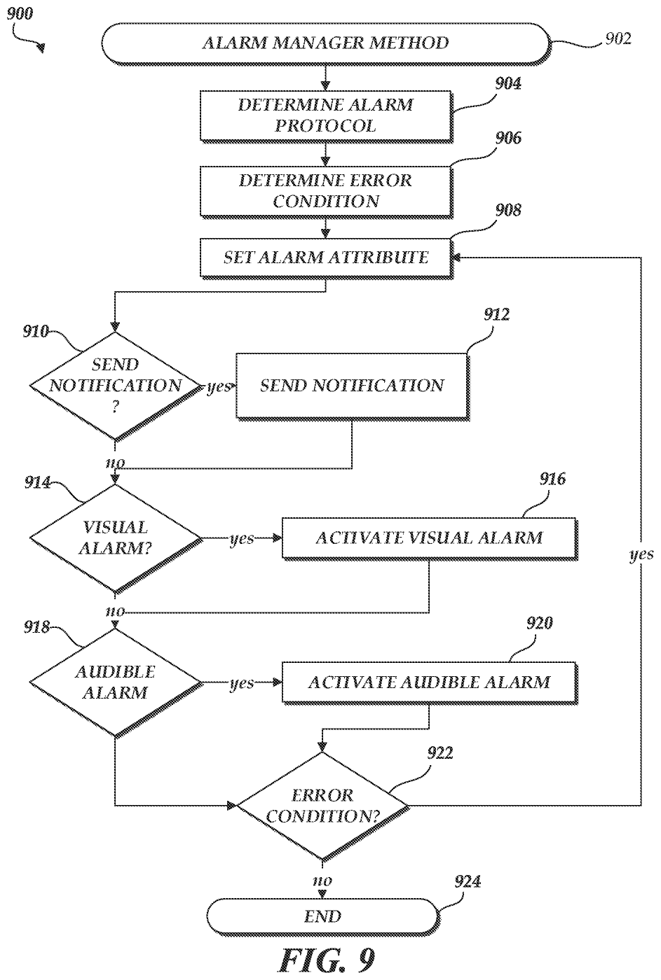

FIG. 9 is a block diagram of one embodiment of a method that can be performed by any of the infusion pump alarm managers described herein. The method 900 begins at block 902. At block 904, the alarm manager determines an alarm protocol. The alarm protocol may be determined from alarm configuration information, including alarm limit data, etc., received from a drug library database. At block 906, the alarm manager determines an error condition. The error condition can correspond to any error state of the infusion pump, including, but not limited to detection of a distal occlusion, the completion of an infusion (e.g., a VTBI complete), etc. At block 908, the alarm manager sets one or more alarm attributes (criticality, priority, audible active, visual active, etc.) for one or more alarm periods, based upon the alarm protocol and the detected error condition.

At block 910, the alarm manager determines whether the alarm protocol requires a notification to be sent during the current alarm period. If yes, the alarm manager sends the notification at block 912. For example, the notification may be sent to an AFS, or directly to an escalation manager, nurses' station, etc. The notification can include an alarm signal that indicates the alarm attributes. At block 914, the alarm manager determines whether to activate a visual alarm on the infusion pump. The visual alarm can include a message, icon and/or image on a display of the infusion pump and/or illumination of an LED or other light or indicator on the infusion pump. If yes, the alarm manager activates the visual alarm at block 916. At block 918, the alarm manager determines whether to activate an audible alarm on the infusion pump. If yes, the alarm manager activates the audible alarm at block 920.

At block 922 the alarm manager determines whether the error condition has persisted. For example, the alarm manager may determine whether the error condition has self-corrected or not. The alarm manager may monitor the presence of the error condition for a first predetermined alarm period. The duration of the first predetermined alarm period may be defined by the alarm protocol. If the error condition is no longer present, the method proceeds to ends at block 924. If the error condition is still present at the end of the first predetermined alarm period, the method may enter a subsequent (e.g., second) alarm period, and return to block 908. At block 908, the alarm attributes are set or updated. For example, the alarm criticality attribute may be updated from LOW to MEDIUM, from MEDIUM to HIGH, from HIGH to CRITICAL, etc. The alarm attributes may include any of the alarm attributes described herein. The alarm manager then proceeds through and repeats blocks 908-922 until the error condition is addressed.

### Other Considerations

It is to be understood that not necessarily all objects or advantages may be achieved in accordance with any particular embodiment described herein. Thus, for example, those skilled in the art will recognize that certain embodiments may be configured to operate in a manner that achieves or optimizes one advantage or group of advantages as taught herein without necessarily achieving other objects or advantages as may be taught or suggested herein.

Many other variations than those described herein will be apparent from this disclosure. For example, depending on the embodiment, certain acts, events, or functions of any of the algorithms described herein can be performed in a different sequence, can be added, merged, or left out altogether (e.g., not all described acts or events are necessary for the practice of the algorithms) Moreover, in certain embodiments, acts or events can be performed concurrently, e.g., through multi-threaded processing, interrupt processing, or multiple processors or processor cores or on other parallel architectures, rather than sequentially. In addition, different tasks or processes can be performed by different machines and/or computing systems that can function together.

The various illustrative logical blocks, modules, and algorithm elements described in connection with the embodiments disclosed herein can be implemented as electronic hardware, computer software, or combinations of both. To clearly illustrate this interchangeability of hardware and software, various illustrative components, blocks, modules, and elements have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The various illustrative logical blocks and modules described in connection with the embodiments disclosed herein can be implemented or performed by a machine, such as a processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor can be a microprocessor, but in the alternative, the processor can be a controller, microcontroller, or state machine, combinations of the same, or the like. A processor can include electrical circuitry configured to process computer-executable instructions. In another embodiment, a processor includes an FPGA or other programmable device that performs logic operations without processing computer-executable instructions. A processor can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor may also include primarily analog components. For example, some or all of the signal processing algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of a method, process, or algorithm described in connection with the embodiments disclosed herein can be embodied directly in hardware, in a software module stored in one or more memory devices and executed by one or more processors, or in a combination of the two. A software module can reside in RAM memory, flash memory, ROM memory, EPROM memory, EEPROM memory, registers, hard disk, a removable disk, a CD-ROM, or any other form of non-transitory computer-readable storage medium, media, or physical computer storage known in the art. An example storage medium can be coupled to the processor such that the processor can read information from, and write information to, the storage medium. In the alternative, the storage medium can be integral to the processor. The storage medium can be volatile or nonvolatile. The processor and the storage medium can reside in an ASIC. The ASIC can reside in a user terminal. In the alternative, the processor and the storage medium can reside as discrete components in a user terminal.

Conditional language used herein, such as, among others, “can,” “might,” “may,” “e.g.,” and the like, unless specifically stated otherwise, or otherwise understood within the context as used, is generally intended to convey that certain embodiments include, while other embodiments do not include, certain features, elements, and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

Unless otherwise explicitly stated, articles such as “a”, “an”, or “the” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B, and C” can include a first processor configured to carry out recitation A working in conjunction with a second processor configured to carry out recitations B and C.

While the above detailed description has shown, described, and pointed out novel features as applied to various embodiments, it will be understood that various omissions, substitutions, and changes in the form and details of the devices or algorithms illustrated can be made without departing from the spirit of the disclosure. As will be recognized, certain embodiments described herein can be implemented within a form that does not provide all of the features and benefits set forth herein, as some features can be used or practiced separately from others. All such modifications and variations are intended to be included herein within the scope of this disclosure. Further, additional embodiments created by combining any two or more features or techniques of one or more embodiments described herein are also intended to be included herein within the scope of this disclosure.

What is claimed is:

1. An infusion pump configured to execute one or more alarm actions according to an alarm protocol, the infusion pump comprising:
a processor; and
a memory in communication with the processor and configured to store instructions that when executed by the processor cause the execution of an alarm manager configured to:
receive alarm configuration information comprising one or more alarm protocols, each alarm protocol comprising one or more alarm parameters;
determine the presence of an error condition; and
perform an alarm action associated with the one or more alarm parameters in response to determining the presence of the error condition.

2. The infusion pump of claim 1, wherein each alarm protocol comprises one or more context parameters associated with each alarm parameter.

3. The infusion pump of claim 2, wherein the context parameters comprise one or more of a drug identifier, an infusate identifier, a therapy identifier, a concentration, or a clinical care area.

4. The infusion pump of claim 1, wherein the alarm parameters comprise one or more alarm limits, alarm types, alarm rules, or alarm behaviors.

5. The infusion pump of claim 1, wherein the alarm parameters comprise one or more of an alarm priority, an alarm criticality, or alarm audio information.

6. The infusion pump of claim 1, wherein the alarm protocol defines a period during which a visual alarm on the infusion pump is activated, and an audible alarm on the infusion pump is not activated.

7. The infusion pump of claim 1, wherein the alarm protocol defines a period during which a visual alarm is activated, an audible alarm is not activated, and an alarm signal indicative of an alarm attribute corresponding to a priority level of the alarm attribute is transmitted to an alarm forwarding system.

8. The infusion pump of claim 1, wherein the alarm protocol defines a period during which a visual alarm is activated, an audible alarm is activated, and an alarm signal indicative of an alarm attribute corresponding to a priority level of the alarm attribute is transmitted to an alarm forwarding service.

9. The infusion pump of claim 1, wherein the alarm manager is further configured to receive the alarm configuration information from a drug library.

10. The infusion pump of claim 1, wherein the alarm manager is further configured to escalate an alarm by changing an alarm attribute and communicating the alarm attribute to an alarm forwarding system.

11. A method of managing alarms of an infusion pump according to an alarm protocol, comprising:
receiving alarm configuration information comprising one or more alarm protocols, each alarm protocol comprising one or more alarm parameters;
determining the presence of an error condition; and
performing an alarm action associated with the one or more alarm parameters in response to determining the presence of the error condition.

12. The method of claim 11, wherein each alarm protocol comprises one or more context parameters associated with each alarm parameter.

13. The method of claim 12, wherein the context parameters comprise one or more of a drug identifier, an infusate identifier, a therapy identifier, a concentration, or a clinical care area.

14. The method of claim 11, wherein the alarm parameters comprise one or more alarm limits, alarm types, alarm rules, or alarm behaviors.

15. The method of claim 11, wherein the alarm parameters comprise one or more of an alarm priority, an alarm criticality, or alarm audio information.

16. The method of claim 11, further comprising, during a period, activating a visual alarm on the infusion pump and not activating an audible alarm on the infusion pump.

17. The method of claim 11, further comprising, during a period, activating a visual alarm on the infusion pump, not activating an audible alarm on the infusion pump, and transmitting an alarm signal indicative of an alarm attribute corresponding to a priority level of the alarm attribute to an alarm forwarding system.

18. The method of claim 11, further comprising, during a period, activating a visual alarm on the infusion pump, activating an audible alarm on the infusion pump, and transmitting an alarm signal indicative of an alarm attribute corresponding to a priority level of the alarm attribute to an alarm forwarding service.

19. The infusion pump of claim 11, wherein receiving the alarm configuration information comprises receiving the alarm configuration information from a drug library.

20. The infusion pump of claim 11, further comprising escalating an alarm by changing an alarm attribute and communicating the alarm attribute to an alarm forwarding system.
